(19) United States
(12) Patent Application Publication
(10) Pub. No.: US 2026/0311838 A1
(43) Pub. Date: Oct. 8, 2026

(54) METHOD AND SYSTEM FOR CARRYING OUT A COMPUTERIZED HYBRID THERAPY SESSION INVOLVING BOTH HUMAN AND VIRTUAL THERAPISTS

(71) Applicant: Blue Mullet LLC, BALTIMORE, MD (US)

(72) Inventor: Yoram FELDMAN, Ramat Gan (IL)

(73) Assignee: Blue Mullet LLC, BALTIMORE, MD (US)

(21) Appl. No.: 19/403,900

(22) Filed: Nov. 30, 2025

Related U.S. Application Data
(63) Continuation of application No. 19/169,014, filed on Apr. 3, 2025, now abandoned.

(51) Int. Cl. G16H 20/30; G06T 13/40; G16H 40/67; G16H 80/00

(52) U.S. Cl. CPC G16H 20/30; G06T 13/40; G16H 40/67; G16H 80/00

(57) ABSTRACT

A system and method of monitoring at least one patient practicing exercises, using a virtual therapist are provided herein. The method may include the following steps: obtaining patient data for the patient; presenting to the patient the virtual therapist, which instructs exercises; monitoring, using sensors and a computer processor, a performance of the exercises by the patient and virtual interaction which includes an interaction between the patient and the virtual therapist; applying the performance and virtual interaction, to a machine learning model with has been trained on datasets associated with patient data, to yield feedback on the performance and on the virtual interaction. The method further includes the step of: upon detecting a deviation from a score of the performance and a score of the virtual interaction, presenting the at least one human therapist via the user interface, in addition to the virtual therapist, to establish a hybrid session.

### CROSS REFERENCE TO RELATED APPLICATIONS

This application is a Continuation of U.S. patent application Ser. No. 19/169,014, filed on Apr. 3, 2025, which is incorporated herein by reference in its entirety.

### FIELD OF THE INVENTION

The present invention relates generally to the field of computer-aided therapy, and more particularly, to carrying out computer-aided therapy sessions involving both human and virtual therapists.

### BACKGROUND OF THE INVENTION

Computer-aided therapy, such as physiotherapy, has undergone extensive progress over the last decade. More and more computer platforms are offering now therapy sessions in which a patient is provided with videos and/or video games showing him or her exercises which were either prescribed by human therapists or computer-generated.

During these sessions, a setup of a camera, computer processor and possibly other sensors, and designated software are arranged to monitor the movements of the patients as they carry out the exercises. Then, using machine learning and visual analysis, an assessment of the exercises carried out by the patient is generated.

The assessment produced by currently available technology is usually a quantitative indication of how well the exercises were performed. For example, the degree of deviation for every movement and accuracy of the movements in terms of percentage from prescribed movements may be monitored. As a result, feedback to perfect the joints angles may be provided. The assessment may be delivered as a score to either the human therapist or the patient or both.

The problem with currently available technology is that these computerized platforms lack the human-like engagement which characterizes therapy sessions with a human therapist. In reality, these platforms are not effective enough in driving patients to reach their full potential in therapy but rather create frustration for not achieving the quantitative goal, presented as a number of values, and as a result lead to lack of adherence to the exercises plan.

Therefore, one of the main challenges in this field is how to provide the patient with a therapy experience that resembles as much as possible, a therapy experience provided by a human therapist. That includes paying attention to the overall cognitive and physical condition of the patient in general, in the current session and in the current exercise.

### SUMMARY OF THE INVENTION

In order to address the aforementioned challenge, some embodiments of the present invention improve the basic technology of computer-aided therapy by providing a computerized platform which may establish a hybrid session by inviting a human therapist to join the virtual therapist on a so-called hybrid therapy session. Embodiments of the present invention may be implemented using machine learning techniques which derive insights from the interaction during the hybrid session.

Some embodiments of the present invention provide a computerized platform with a virtual therapist implemented by a model which is capable of identifying the need to call on a human therapist into the session to debate on a situation. In such a hybrid session, both therapists may be present and discussing the situation.

In more detail, embodiments of the present invention provide a computer-implemented method of monitoring at least one patient practicing exercises, using at least one virtual therapist. The method may include the following steps: obtaining, on a computer memory, patient data for the at least one patient, the patient data may include description of physical profile of the at least one patient; presenting to the at least one patient, via a user interface, the at least one virtual therapist, which instructs exercises, monitoring, using sensors and a computer processor, a performance of the exercises and/or games by the at least one patient and virtual interaction which includes an interaction between the at least one patient and the virtual therapist; applying, via the computer processor, the performance and virtual interaction, to a machine learning model with has been trained on datasets associated with patient data, to yield feedback on the patient performance and on the virtual interaction.

The method further includes the step of, upon detecting, by the computer processor, a deviation from a score of the performance and a score of the virtual interaction, presenting the at least one human therapist via the user interface, in addition to the virtual therapist, to create a hybrid session; monitoring using the sensors and the computer processor, during the hybrid session: the performance, a hybrid interaction which comprises: the interaction between the patient and the virtual therapist, an interaction between the patient and the at least one human therapist, and an interaction between the at least one human therapist and the virtual therapist; training the machine learning model based on the hybrid interaction and the performance during the hybrid session, to improve an accuracy of the machine learning model; and switching back to the virtual session, responsive to an indication by the least one human therapist.

Advantageously, embodiments of the present invention greatly improve the user's experience of the therapy session. It improves the engagement of the patient with the therapy process and also improves the accuracy of the model by which the virtual therapist is operating, which benefits the therapy of the particular patient as well as other patients using the same platform.

On the human therapist side, embodiments of the present invention improve timely involvement in a session as it reduces the inefficient sessions thanks to the early detection of the need for their involvement. Furthermore, it improves the trust of the human therapist in the virtual therapist and as a result speeds up the adoption of virtual therapists to the benefit of the entire healthcare ecosystem.

These and other advantages of the present invention are set forth in detail in the following description

### BRIEF DESCRIPTION OF THE DRAWINGS

The subject matter regarded as the invention is particularly pointed out and distinctly claimed in the concluding portion of the specification. The invention, however, both as to organization and method of operation, together with objects, features, and advantages thereof, may best be understood by reference to the following detailed description when read with the accompanying drawings in which:

FIG. 1 is a block diagram illustrating a system for computerized aided platforms capable of orchestrating a hybrid therapy session involving a patient, a virtual therapist, and a human therapist who appears upon demand, in accordance with embodiments of the present invention;

FIG. 2A and FIG. 2B show a flowchart illustrating a method implementing the aforementioned system, in accordance with some embodiments of the present invention; and

FIG. 3 is a block diagram illustrating a system for computerized aided platforms implementing embodiments of the present invention in client-server configuration.

It will be appreciated that for simplicity and clarity of illustration, elements shown in the figures have not necessarily been drawn to scale. For example, the dimensions of some of the elements may be exaggerated relative to other elements for clarity.

Further, where considered appropriate, reference numerals may be repeated among the figures to indicate corresponding or analogous elements.

### DETAILED DESCRIPTION OF THE INVENTION

In the following description, various aspects of the present invention will be described. For purposes of explanation, specific configurations and details are set forth in order to provide a thorough understanding of the present invention. However, it will also be apparent to one skilled in the art that the present invention may be practiced without the specific details presented herein. Furthermore, well-known features may be omitted or simplified in order not to obscure the present invention.

Unless specifically stated otherwise, as apparent from the following discussions, it is appreciated that throughout the specification discussions utilizing terms such as “processing”, “computing”, “calculating”, “determining”, or the like, refer to the action and/or processes of a computer or computing system, or similar electronic computing device, that manipulates and/or transforms data represented as physical, such as electronic, quantities within the computing system's registers and/or memories into other data similarly represented as physical quantities within the computing system's memories, registers or other such information storage, transmission or display devices.

In the foregoing detailed description, numerous specific details are set forth in order to provide an understanding of the invention. However, it will be understood by those skilled in the art that the invention can be practiced without these specific details. In other instances, well-known methods, procedures, and components, modules, units, and/or circuits have not been described in detail so as not to obscure the invention. Some features or elements described with respect to one embodiment can be combined with features or elements described with respect to other embodiments.

In some embodiments, the hybrid session may be in the form of a consultation where the virtual therapist explains to the human therapist the condition and the state of the exercises in the session and seeks his or her advice. During the hybrid session, sensors which may include an exemplary non-limiting set-up of a camera, microphone, proximity sensors, computer processor and designated software may be arranged to use machine learning, to train the model according to which the virtual therapist interacts with the patient.

In more detail, embodiments of the present invention include a method of monitoring, using a computerized platform, a patient exercising a series of exercises and/or video games prescribed by a human therapist or the virtual therapist, using a virtual therapist. During session, the patient interacts with the virtual therapist, which in turn provides verbal feedback to the patient.

Whenever the computer processor which controls the computerized platform detects a deviation in the patient performance and/or in the interaction between the patient and the virtual patient, a human therapist is invited into the session possibly but not necessarily on the respective electronic display in addition to the virtual therapist, to join and create a hybrid session.

Embodiments of the present invention enable monitoring, using sensors and the computer processor, during the hybrid session, the patient performance, and the following interactions:
1. Interaction between the patient and the virtual therapist;
2. Interaction between the patient and the human therapist; and
3. Interaction between the human therapist and the virtual therapist;

Embodiments of the present invention use the aforementioned monitoring in order to further training the machine learning model, during the hybrid session to improve the accuracy of the machine learning model.

Embodiments of the present invention are capable of switching back the computerized platform into the virtual therapist once the hybrid training session has ended, responsive to indication by the human therapist that the hybrid session is over.

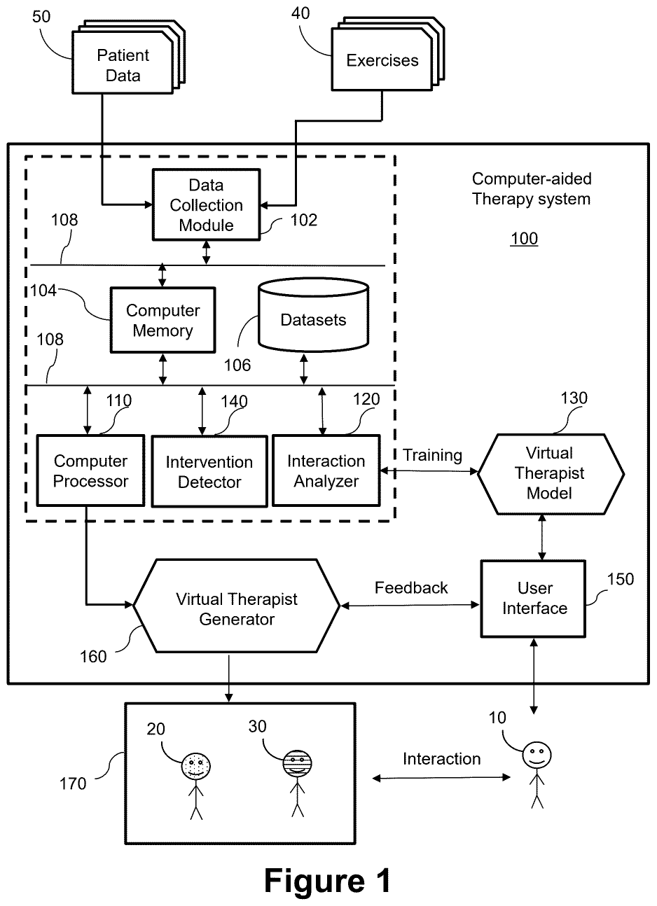

FIG. 1 is a high-level block diagram illustrating a computer-aided therapy system 100 for monitoring at least one patient 10 practicing exercises such as physiotherapy exercises, using at least one virtual therapist 20. System 10 may include: a computer memory 104 connected via a bus 108 to a data collection module 102, for obtaining patient data 50 for the at least one patient 10, wherein patient data 50 may include a description of physical condition of the patient 10 including his or her therapy history, performance and capabilities. System 100 may further include a user interface 150, for presenting to the at least one patient 10, the at least one virtual therapist 20, which instructs exercises prescribed to the at least one patient 10.

According to some embodiments of the present invention, System 100 may further include sensors, as part of the user interface 150 and a computer processor 110 connected via bus 108 to computer memory 104 which are used for monitoring a performance of the exercises as they are being executed by the at least one patient 10 and virtual interaction which may include an interaction between the at least one patient 10 and the virtual therapist 20. This may be carried out by interaction analyzer 120 implemented by computer processor 110.

According to some embodiments of the present invention, user interface 150 may include a three-dimensional humanoid robot taking the form of virtual therapist 20 and being capable of demonstrating the exercises to patient 150. Humanoid robot user interface 150 may also apply some physiotherapy treatment to patient 10 in line with the prescribed exercises. The session carried out via humanoid robot user interface 150 may be monitored by the sensors and data relating to the interaction may be used in updating the machine learning model.

According to some embodiments of the present invention, computer processor 110 may be used for applying, the performance and virtual interaction, to a machine learning model such as virtual therapist model 130 with has been trained on datasets 106 associated with patient data 50, to yield feedback on the patient performance and on the virtual interaction.

According to some embodiments of the present invention, upon detecting, by computer processor 110 possibly using intervention analyzer 140 in communication with computer processor 110 via bus 108, a deviation from a score of the performance and a score of the virtual interaction, computer processor 110 may be configured to invite the at least one human therapist 30 onto user interface 150, in addition to the virtual therapist 20, to create a hybrid session.

According to some embodiments of the present invention, the sensors in user interface 150 and computer processor 110 are configured, during the hybrid session to monitor: the performance, a hybrid interaction which may include: the interaction between the patient and the virtual therapist, an interaction between the patient and the at least one human therapist, and an interaction between the at least one human therapist and the virtual therapist.

According to some embodiments of the present invention, computer processor 110 may be configured to train the machine learning model of virtual therapist model 130 based on the hybrid interaction and the performance during the hybrid session, to improve the accuracy of virtual therapist model 130.

According to some embodiments of the present invention, computer processor 110 is configured to switch back to the virtual session, responsive to an indication by the least one human therapist.

According to some embodiments of the present invention, the sensors comprise at least one of; a camera and a microphone.

According to some embodiments of the present invention, virtual therapist 20 may be generated based on virtual therapist model 120 by virtual therapist generator 160 in communication with an electronic display 170 for generating graphics of a human-like object which use lip syncing based on text that has been provided and text-to-speech providing a notion of an avatar.

According to some embodiments of the present invention, the avatar representation of the virtual therapist presented on electronic display 170 may provide demonstrations on how to perform the exercises. This may be in addition or instead of the videos showing the exercises tailored for patient 10.

According to some embodiments of the present invention, the presenting by virtual therapist generator 160 and electronic display 170 of virtual therapist 20 may include generating video featuring virtual therapist 20 following real-life videos from a database of exercises tailored to the physical condition of patient 10.

According to some embodiments of the present invention, computer processor 110 and user interface 150 are configured to display to patient 10, videos with mark-ups on the non-compliant performance of the patient.

According to some embodiments of the present invention, patient data 50 further includes history of treatment of patient 10.

According to some embodiments of the present invention, the machine learning model of virtual therapist model 130 may include large language model (LLM) which reacts to the hybrid interaction.

According to some embodiments of the present invention, the machine learning model of virtual therapist model 130 which may be trained during the hybrid session is used to improve the exercise demonstration by the virtual therapist.

According to some embodiments of the present invention the presenting by the virtual therapist comprises generating video featuring the virtual therapist following real-life videos from a database of exercises tailored to the patient profile.

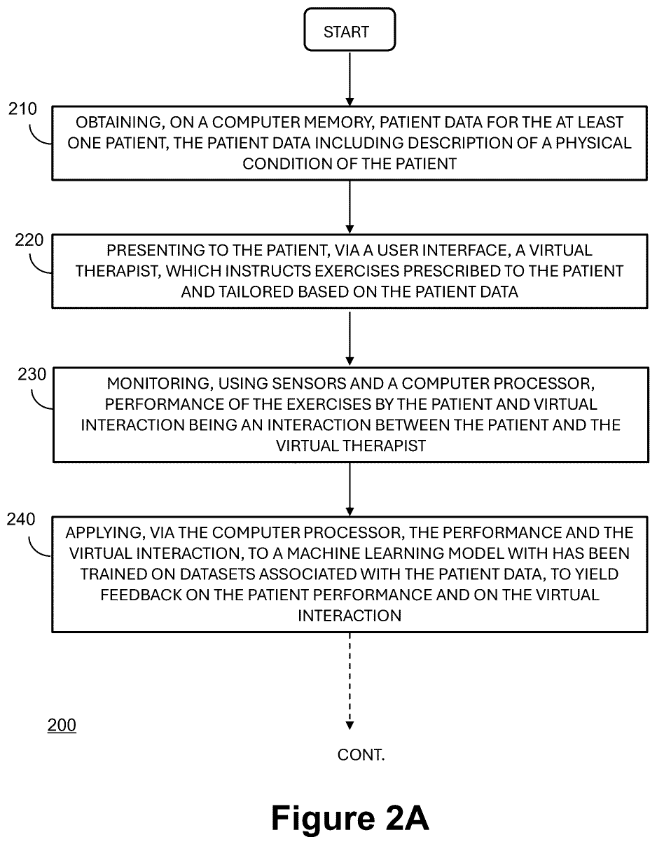

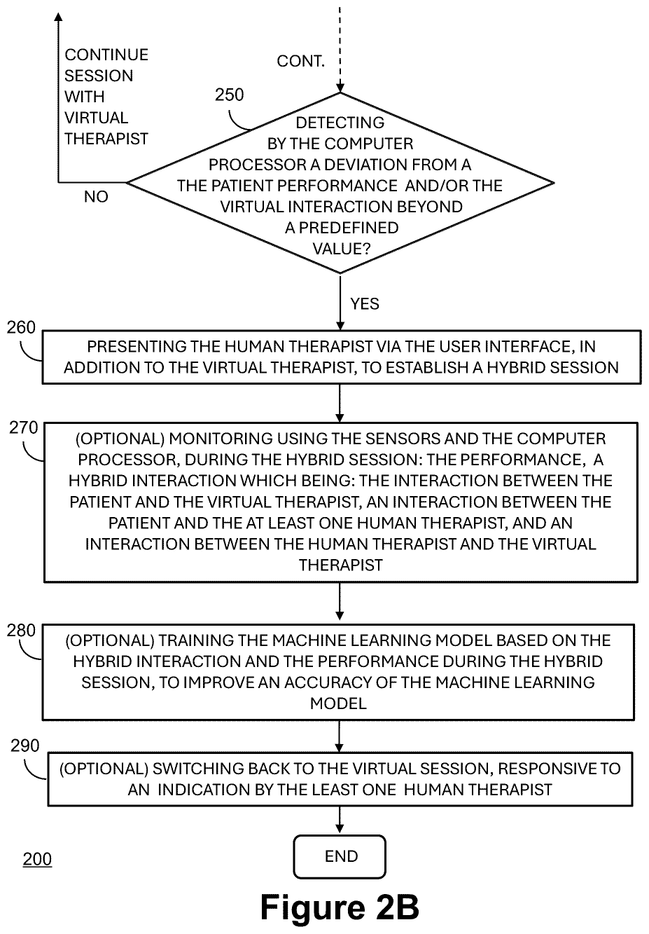

FIG. 2 is a high-level flowchart illustrating computer-implemented method 200 of monitoring at least one patient practicing exercises using at least one virtual therapist. Method 200 may include the following steps: obtaining, on a computer memory, patient data of the patient, said patient data comprising a physical profile of the patient 210; presenting to the patient, via a user interface, the virtual therapist, which instructs exercises prescribed to the at least one patient 220; monitoring, using sensors and a computer processor, performance of the exercises by the patient and a virtual interaction comprising an interaction between the patient and the virtual therapist 230; applying the performance and the virtual interaction to a machine learning model implemented on the computer processor which has been trained on datasets associated with patient data, to yield feedback on the performance and on the virtual interaction 240; upon detecting 250 by the computer processor, a deviation from a score of the performance and a score of the virtual interaction, inviting a human therapist, in addition to the virtual therapist, establishing, over the via the user interface, a hybrid session which includes both the virtual therapist 260; optionally, method 200 may further include the step of monitoring using the sensors and the computer processor, during the hybrid session: the performance, a hybrid interaction which comprises at least one of: the interaction between the patient and the virtual therapist, an interaction between the patient and the human therapist, and an interaction between the human therapist and the virtual therapist 270; Optionally, method 200 may further include the step training the machine learning model based on the hybrid interaction and the performance during the hybrid session, to improve an accuracy of the machine learning model 280; Further optionally, method 200 may further include the step switching back to the virtual session, responsive to an indication by the human therapist 290.

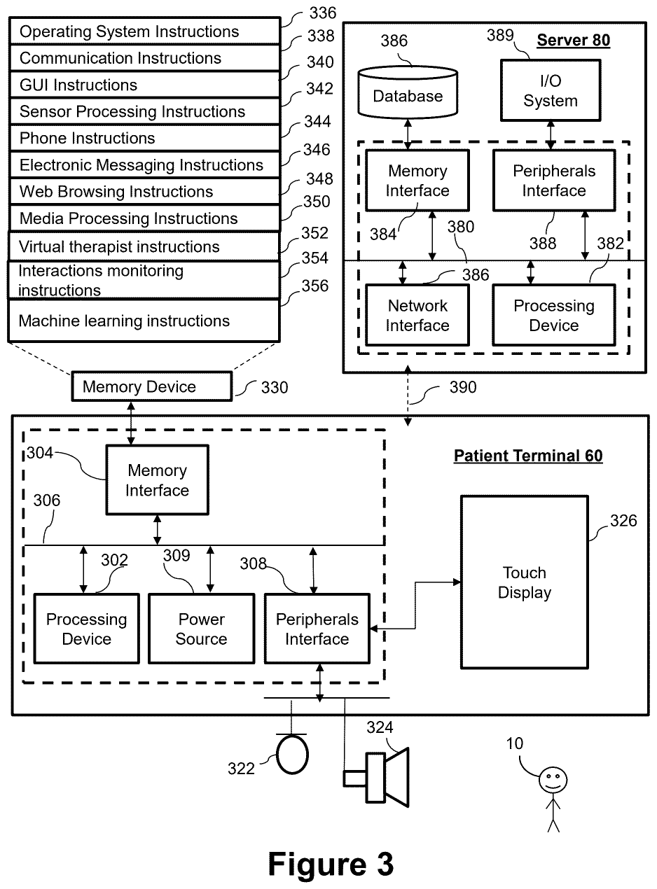

FIG. 3 shows a block diagram of the configuration of a patient device 60 and a server 80 to serve as a system for monitoring session with a virtual therapist. With regard to patient terminal 60, and according to some embodiments, patient terminal 60, directly or indirectly, may access a bus 306 (or another data transfer mechanism) that interconnects subsystems and components for transferring information within the patient terminal 60. For example, bus 306 may interconnect a processing device 302, a memory interface 304, and a peripherals interface 308 connected to an I/O system such as display 326.

In some embodiments, processing device 302 may use a memory interface 304 to access data and a software product stored on a memory device 330.

According to some embodiments, the peripherals interface 308 may also be connected to sensors, devices, and subsystems to facilitate multiple functionalities. In one embodiment, the peripherals interface 308 may be connected to an I/O system such as display 326 configured to receive signals or input from devices and to provide signals or output to one or more devices that allow data to be received and/or transmitted by the patient terminal 60. In one example, the I/O system 326

According to some embodiments, the peripherals interface 208 may also be connected to an image sensor 226, a motion sensor 228, a light sensor 230, and/or a proximity sensor 232 to facilitate image capturing, orientation, lighting, and proximity functions. In addition, a GPS receiver may also be integrated with, or connected to, the patient terminal 20, such as GPS receivers typically integrated into mobile communications devices. Alternatively, GPS software may permit the patient terminal 20 to access an external GPS receiver (e.g., connecting via a serial port or Bluetooth).

The memory device 330 may store operating system instructions 336 to facilitate managing the operating system of patient terminal 60, communication instructions 338 to facilitate communicating with one or more additional devices, one or more computers, and/or one or more servers. The memory device 330 may include: graphical user interface instructions 340 to facilitate graphic user interface processing; sensor processing instructions 342 to facilitate sensor-related processing and functions; phone instructions 344 to facilitate phone-related processes and functions; electronic messaging instructions 346 to facilitate electronic-messaging-related processes and functions; web browsing instructions 348 to facilitate web browsing-related processes and functions; media processing instructions 350 to facilitate media processing-related processes and functions; virtual therapist instructions 352 to facilitate interactions monitoring instructions 354; and machine learning instructions 354 to facilitate processes and functions related to training the virtual therapist model.

Each of the above-identified instructions and applications may correspond to a set of instructions for performing one or more functions described above. These instructions do not necessarily need to be implemented as separate software programs, procedures, or modules. The memory device 330 may include additional instructions or fewer instructions. Furthermore, various functions of patient terminal 60 may be implemented in hardware and/or software, including in one or more signal processing and/or application-specific integrated circuits.

Still referring to FIG. 3, and according to some embodiments of the present invention, a server 80 for monitoring physiotherapy session carried out by a virtual therapist with a patient via on at least one patient terminal 60, is illustrated herein.

Processing device 382 may include at least one processor configured to execute computer programs, applications, methods, processes, or other software to perform embodiments described in the present disclosure.

In some embodiments, processing device 382 may use a memory interface 382 to access data and a software product stored on a memory device or a non-transitory computer-readable medium or to access a database 386.

According to some embodiments, the network interface 386 may provide two-way data communication to a network. In FIG. 3, the communication 390 between patient terminal 60 and server 80 is represented by a dashed arrow. In one embodiment, the network interface 386 may include an integrated services digital network card, cellular modem, satellite modem, or a modem to provide a data communication connection over the Internet. As another example, the network interface 386 may include a wireless local area network (WLAN) card. In another embodiment, the network interface 386 may include an Ethernet port connected to radio frequency receivers and transmitters. The specific design and implementation of the network interface 386 may depend on the communications network(s) over which the patient terminal 60 and the server 80 may communicate.

According to some embodiments, the server 80 may also include a peripherals interface 388 coupled to the bus 380. The peripherals interface 388 may also be connected to devices, and subsystems to facilitate multiple functionalities as performed by the server 80. In some embodiments, those devices and subsystems may comprise a display screen (e.g., LCD) a USB port, and the like.

According to some embodiments interaction monitoring instructions 354, when executed on the computer processor of processing device 302 are monitoring using the sensors and the computer processor, during the hybrid session: the performance, a hybrid interaction which comprises: the interaction between the patient and the virtual therapist, an interaction between the patient and the at least one human therapist, and an interaction between the at least one human therapist and the virtual therapist.

According to some embodiments machine learning instructions 356, when executed on the computer processor of processing device 302 are training the machine learning model based on the hybrid interaction and the performance during the hybrid session, to improve an accuracy of the machine learning model; and determine the switching back to the virtual session, responsive to an indication by the least one human therapist.

The components and arrangements shown in FIG. 3 for both the server 80 and the patient terminal 60 are not intended to limit the disclosed embodiments. As will be appreciated by a person skilled in the art having the benefit of this disclosure, numerous variations and/or modifications may be made to the depicted configuration of the server 80 and the patient terminal 20. For example, not all the depicted components may be essential for the operation of server 80 or the patient terminal 60 in all cases. Any component may be located in any appropriate part of the server 80, and the components may be rearranged into a variety of configurations while providing the functionality of the disclosed embodiments. For example, some types of patient terminals 20 may include only part of the depicted sensors and include sensors which are not depicted.

It is further understood that some embodiments of the present invention may be embodied in the form of a system, a method, or a computer program product. Similarly, some embodiments may be embodied as hardware, software, or a combination of both. Some embodiments may be embodied as a computer program product saved on one or more non-transitory computer-readable medium in the form of computer-readable program code embodied thereon. Such non-transitory computer-readable medium may include instructions that when executed cause a processor to execute method steps in accordance with embodiments. In some embodiments, the instructions stored on the computer-readable medium may be in the form of an installed application and in the form of an installation package.

Such instructions may be, for example, loaded by one or more processors and executed. For example, the computer-readable medium may be a non-transitory computer-readable storage medium. A non-transitory computer-readable storage medium may be, for example, an electronic, optical, magnetic, electromagnetic, infrared, or semiconductor system, apparatus, or device, or any combination thereof.

Computer program code may be written in any suitable programming language. The program code may execute on a single computer system, or on a plurality of computer systems.

One skilled in the art will realize the invention may be embodied in other specific forms without departing from the spirit or essential characteristics thereof. The foregoing embodiments are therefore to be considered in all respects illustrative rather than limiting of the invention described herein. Scope of the invention is thus indicated by the appended claims, rather than by the foregoing description, and all changes that come within the meaning and range of equivalency of the claims are therefore intended to be embraced therein.

In the foregoing detailed description, numerous specific details are set forth in order to provide an understanding of the invention. However, it will be understood by those skilled in the art that the invention can be practiced without these specific details. In other instances, well-known methods, procedures, and components, modules, units, and/or circuits have not been described in detail so as not to obscure the invention. Some features or elements described with respect to one embodiment can be combined with features or elements described with respect to other embodiments.

1. A system for monitoring a patient practicing exercises, using a virtual therapist, the system comprising:
a computer memory arranged to obtain patient data for the patient, said patient data comprising a physical profile of the patient;
a user interface arranged to present to the patient, the virtual therapist, which instructs exercises prescribed to the patient;
a plurality of sensors proximal to the patient; and
a computer processor arranged to:
receive measurements of the sensor and monitor performance of the exercises carried out by the patient and a virtual interaction comprising an interaction between the at patient and the virtual therapist; and
apply the performance and the virtual interaction to a machine learning model implemented on the computer processor, which has been trained on datasets associated with patient data, to yield feedback on the performance and on the virtual interaction,
wherein upon detecting, by the computer processor, of a deviation from a score of the performance and a score of the virtual interaction, the computer processor is further arranged to:
invite a human therapist, in addition to the virtual therapist, to establish, over the via the user interface, a hybrid session which includes both the virtual therapist and the human therapist;
monitor based on the measurements of the sensors, during the hybrid session: the performance and a hybrid interaction which comprises at least one of: the interaction between the patient and the virtual therapist, the interaction between the patient and human therapist, and the interaction between the at least one human therapist and the virtual therapist;
train the machine learning model based on the hybrid interaction and the performance during the hybrid session, to improve an accuracy of the machine learning model; and
switch back to the virtual session, responsive to an indication by the human therapist.

2. The system according to claim 1, wherein the sensors comprise at least one of: a camera, a microphone, a keyboard, a touchscreen, and pen pad.

3. The system according to claim 1, wherein the user interface comprises at least one of: an electronic display, and a speaker.

4. The system according to claim 1, wherein the user interface comprises a display and wherein the virtual therapist has a human-like appearance and further uses lip syncing based on text that has been provided and text-to-speech providing a notion of an avatar.

5. The system according to claim 1, wherein the user interface comprises a three-dimensional humanoid robot capable of demonstrating the exercises to the at least one patient.

6. The system according to claim 1, wherein the exercises are in a form of at least one of: video game, a computer game.

7. The system according to claim 1, wherein the patient profile comprises at least one of: capabilities of the patient and performance of the patient.

8. The system according to claim 1, wherein said machine learning model comprises large language model (LLM) which is arranged to react to at least one of: the virtual interaction; and
the hybrid interaction.

9. The system according to claim 1, wherein the computer processor is further arranged to use the machine learning model trained during the hybrid session to improve the exercises presented by the virtual therapist.

10. A method of monitoring a patient practicing exercises, using a virtual therapist, the method comprising:
obtaining, on a computer memory, patient data of the patient, said patient data comprising a physical profile of the patient;
presenting to the patient, via a user interface, the virtual therapist, which instructs exercises prescribed to the patient;
monitoring, using sensors and a computer processor, performance of the exercises by the patient and a virtual interaction comprising an interaction between the patient and the virtual therapist;
applying the performance and the virtual interaction to a machine learning model implemented on the computer processor which has been trained on datasets associated with patient data, to yield feedback on the performance and on the virtual interaction;
upon detecting, by the computer processor, a deviation from a score of the performance and a score of the virtual interaction, inviting a human therapist, in addition to the virtual therapist, to establish, over the via the user interface, a hybrid session which includes both the virtual therapist;
monitoring using the sensors and the computer processor, during the hybrid session: the performance, a hybrid interaction which comprises at least one of: the interaction between the patient and the virtual therapist, an interaction between the patient and the human therapist, and an interaction between the human therapist and the virtual therapist;
training the machine learning model based on the hybrid interaction and the performance during the hybrid session, to improve an accuracy of the machine learning model; and
switching back to the virtual session, responsive to an indication by the human therapist.

11. The method according to claim 10, wherein the exercises are in a form of at least one of: video game, a computer game.

12. The method according to claim 10, wherein the patient profile comprises at least one of: capabilities of the patient and performance of the patient.

13. The method according to claim 10, wherein the virtual therapist has a human-like appearance and further uses lip syncing based on text that has been provided and text-to-speech providing a notion of an avatar.

14. The method according to claim 10, wherein the patient profile comprises at least one of: capabilities of the patient and performance of the patient.

15. The method according to claim 10, wherein said machine learning model comprises large language model (LLM) which is arranged to reacts to at least one of: the virtual interaction; and the hybrid interaction.

16. The method according to claim 10, further comprising using the machine learning model trained during the hybrid session, to improve the exercises presented by the virtual therapist.

17. A non-transitory computer readable medium for monitoring a patient practicing exercises, using a virtual therapist, said non-transitory computer readable medium comprising a set of instructions that, when executed, cause at least one computer processor to:
obtain patient data of the patient, said patient data comprising a physical profile of the patient;
present to the patient, via a user interface, the virtual therapist, which instructs exercises prescribed to the at least one patient;
monitor, using sensors, performance of the exercises by the patient and a virtual interaction comprising an interaction between the patient and the virtual therapist;
apply the performance and the virtual interaction to a machine learning model which has been trained on datasets associated with patient data, to yield feedback on the performance and on the virtual interaction;
invite a human therapist, in addition to the virtual therapist, to establish, over the via the user interface, a hybrid session which includes both the virtual therapist, upon detecting a deviation from a score of the performance and a score of the virtual interaction, ;
monitor, using the sensors, during the hybrid session: the performance, a hybrid interaction which comprises at least one of: the interaction between the patient and the virtual therapist, an interaction between the patient and the human therapist, and an interaction between the human therapist and the virtual therapist;
training the machine learning model based on the hybrid interaction and the performance during the hybrid session, to improve an accuracy of the machine learning model; and
switch back to the virtual session, responsive to an indication by the human therapist.

18. The non-transitory computer readable medium according to claim 17, wherein the virtual therapist has a human-like appearance and further uses lip syncing based on text that has been provided and text-to-speech providing a notion of an avatar.

19. The non-transitory computer readable medium according to claim 17, wherein said machine learning model comprises large language model (LLM) which is arranged to reacts to at least one of: the virtual interaction; and the hybrid interaction.

20. The non-transitory computer readable medium according to claim 17, further comprising using the machine learning model trained during the hybrid session, to improve the exercises presented by the virtual therapist.
